(19) United States
(12) Patent Application Publication
(10) Pub. No.: US 2009/0305432 A1
(43) Pub. Date: Dec. 10, 2009

(54) Polypeptide Molecular Switch

(76) Inventors: Lance Liotta, Bethesda, MD (US); Emanuel Petricoin, Gainesville, VA (US); David Geho, Oakton, VA (US)

Correspondence Address: FOLEY AND LARDNER LLP;SUITE 500, 3000 K STREET NW, WASHINGTON, DC, 20007, US

(21) Appl. No.: 12/083,678

(22) PCT Filed: Oct. 16, 2006
(86) PCT No.: PCT/US2006/040227

§ 371 Date: Aug. 10, 2009

Related U.S. Application Data
(60) Provisional application No. 60/726,656, filed on Oct. 17, 2005.
(60) Provisional application No. 60/759,575, filed on Jan. 18, 2006.
(60) Provisional application No. 60/817,656, filed on Jun. 30, 2006.

(51) Int. Cl. G01N 33/53; C07K 7/00; C07K 7/06; C12N 9/12; G11C 7/10; H01L 29/772; G01N 27/00

(52) U.S. Cl. 436/501; 530/300; 435/194; 530/327; 365/189.05; 257/253; 422/82.01; 365/151; 257/E29.242

(57) ABSTRACT

A polypeptide can conduct electricity in a closed circuit. Conformational changes in the polypeptide due to posttranslational modifications or ligand binding can effect the conductive properties of the polypeptide which can be measured. In such a closed circuit, a polypeptide having at least one residue capable of reversible modification can be used as a molecular switch. Circuits comprising such molecular switches can be used, for example, in methods for assessing the modification state of a polypeptide, determining the activity of an enzyme of interest, identifying compounds that affect the activity of an enzyme of interest, storing data, detecting the presence of a compound and identifying inhibitors of protein-protein interactions.

This application claims priority to U.S. Provisional Application No. 60/817,656, filed Jun. 30, 2006, U.S. Provisional Patent Application No. 60/759,575, filed Jan. 18, 2006 and U.S. Provisional Application No. 60/726,656, filed Oct. 17, 2005, all of which are hereby incorporated by reference in their entireties.

### BACKGROUND

The following description of the background of the invention is provided simply as an aid in understanding the invention and is not admitted to describe or constitute prior art to the invention.

Proteins are versatile machines that perform diverse tasks within living systems, including anabolism and catabolism of other molecules, maintaining structural integrity, adhesion, and signal transmission. Despite the variety of proteins and tasks that exist, the correlation of conformational shifts in a protein's structure with altered protein behavior is a unifying property of these polymers. Conferral of posttranslational modifications onto amino acid residues is one mechanism for inducing function-altering conformational shifts in a protein's structure. Of particular interest within the biomedical and pharmaceutical research communities is the role of cell-signaling protein phosphorylation in health and disease processes.

In the emerging field of bioelectronics, biological molecules provide a broad platform for integration with electronic elements to create, for example, bioelectronic switches, biosensors, biological computational elements, biofuel cells, and others. Bioelectronic devices capitalize on the electrical properties of biological molecules such as proteins, peptides, DNA, enzymes, and RNA. In this way, such devices can detect biological events by monitoring resistance, conductance, charge transport, impedance, or other measurable electrical properties.

The electrical property most important to these applications is electron transport. In this regard, conformational changes in a protein due to posttranslational modifications or ligand binding can effect the protein's conductive properties. For example, it was demonstrated several decades ago that electron transport within a mature protein, casein, can be altered by the addition of methylglyoxal. Phosphorylation is another modification that shifts a protein's conformation and alters its functional state.

To date, bioelectronic devices utilizing proteins have utilized open circuits, wherein a solution is employed to carry current. Such aqueous-based systems are incompatible with many of today's electronic applications. Thus, there is a strong need for a solid-state, peptide-based circuit.

### SUMMARY

In one embodiment, a closed circuit comprises a molecular switch comprising a polypeptide having at least one residue capable of reversible modification.

In another embodiment, a device for storing data comprises at least one latch comprising a first logic gate and a second logic gate wherein each first and second logic gate comprises a molecular switch comprising a polypeptide having at least one residue capable of a reversible modification.

In another embodiment, a sensor comprises a closed circuit comprising (i) a polypeptide attached to either a source or drain electrode and connected to the other electrode so as to form a closed circuit, wherein the polypeptide undergoes a conformational change upon binding to a ligand, (ii) means for producing an electrical signal; and (iii) means for measuring the conductive properties of the polypeptide.

In still another embodiment, a method for assessing the modification state of a polypeptide comprises attaching the polypeptide to either a source or drain electrode and connecting the polypeptide to the other electrode so as to form a closed circuit, measuring the conductive properties of the polypeptide; and comparing the measured conductive properties to that of one or more control polypeptides.

In one embodiment, a method of determining the activity of an enzyme of interest in a sample comprises adding to the sample a polypeptide having at least one residue susceptible to modification by the enzyme, recovering the polypeptide from the sample, attaching it to a source or drain electrode and connecting the polypeptide to the other electrode so as to form a closed circuit, measuring the conductive properties of the polypeptide and comparing the measured conductive properties to that of one or more control polypeptides.

In another embodiment, a method of determining the activity of an enzyme of interest in a sample comprises exposing the sample to a closed circuit comprising a molecular switch comprising a polypeptide having at least one residue susceptible to modification by the enzyme, wherein the polypeptide is attached to either a source or drain electrode and connected to the other electrode so as to form a closed circuit, measuring the conductive properties of the polypeptide and comparing the measured conductive properties to that of one or more control polypeptides.

In still another embodiment, a method of identifying compounds that affect the activity of an enzyme of interest, comprises (A) exposing the enzyme to a closed circuit comprising a molecular switch comprising a polypeptide having at least one residue susceptible to modification by the enzyme, wherein the polypeptide is attached to either a source or drain electrode and connected to the other electrode so as to form a closed circuit, and measuring the conductive properties of the polypeptide, (B) exposing the enzyme to a test compound, (C) repeating step (A); and (D) comparing the measured conductive properties from steps (A) and (C).

In one embodiment, a method for storing data comprises providing at least one latch comprising a first logic gate and a second logic gate wherein each first and second logic gate comprises a molecular switch comprising a polypeptide having at least one residue capable of a reversible modification.

In another embodiment a method of detecting the presence of a substance in a sample comprises exposing the sample to a sensor comprising a closed circuit comprising (i) a polypeptide that undergoes a conformational change upon binding to a ligand wherein the polypeptide is attached to either a source or drain electrode and connected to the other electrode so as to form a closed circuit, (ii) means for producing an electrical signal; and (iii) means for measuring the conductive properties of the polypeptide; measuring the conductive properties of the polypeptide; and comparing the measured conductive properties to that of one or more control polypeptides.

In still another embodiment, a method for identifying a compound that inhibits binding between a pair of polypeptides, comprises (A) attaching a first polypeptide of the pair to a source or drain electrode and connecting the first polypeptide to the other electrode so as to form a closed circuit; (B) exposing a second polypeptide of the pair to a test compound; (C) exposing the second polypeptide to the attached first polypeptide; (D) measuring the conductive properties of the first polypeptide; and (E) comparing the measured conductive properties to that of one or more control polypeptides.

In one embodiment, there is provided a method for identifying a compound that inhibits binding between a pair of polypeptides, the method comprising (A) attaching a first polypeptide of the pair to a source or drain electrode and connecting the first polypeptide to the other electrode so as to form a closed circuit; (B) exposing the attached first polypeptide to a second polypeptide of the pair; (C) measuring the conductive properties of the first polypeptide; (D) exposing the attached first polypeptide to a test compound; (E) measuring the conductive properties of the first polypeptide; and (F) comparing the measured conductive properties from steps (C) and (E).

Other objects, features and advantages will become apparent from the following detailed description. The detailed description and specific examples are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples where it will be obviously useful to those skilled in the prior art.

### BRIEF DESCRIPTION OF THE DRAWINGS

FIGS. 1A-B provide a graphical representation of a peptide and a phosphorylated peptide.

FIG. 2 provides a graphical representation of a molecular circuit.

FIGS. 3A, 3C, 3E and 3F provide graphs illustrating I-V characteristics of the molecular circuit in FIG. 2. FIGS. 3B and 3D provide graphs illustrating current versus density characteristics of the molecular circuit in FIG. 2.

FIGS. 4A-C provide graphs illustrating inelastic electron tunneling spectroscopy (IETS) of metal-peptide-metal junctions in molecular circuits.

FIG. 5 provides a SEM image of a microsphere junction of a molecular circuit. Long, et al., Applied Physics Letters 86, 153105 (2005).

FIG. 6 provides a graph illustrating I-V characteristics for molecules used in a molecular circuit. Long, et al., Applied Physics Letters 86, 153105 (2005).

FIG. 7 provides a graphical representation of CaM Kinase II undergoing phosphorylation. Hudmon et al, Biochem. J. (2002) 364, 593-611.

FIG. 8 provides a graphical representation of a residue fragment extracted from the regulatory domain of CaM Kinase II. Hudmon et al, Biochem. J. (2002) 364, 593-611.

FIG. 9 provides a graphical model illustrating the site of phosphorylation for CaM Kinase II. Hudmon et al, Biochem. J. (2002) 364, 593-611.

FIG. 10 provides a graphical model of a CaM Kinase II derived peptide.

FIG. 11 provides a graphical model of a phosphorylated CaM Kinase II derived peptide.

FIG. 12 provides a graphical representation of a receptor protein electrically connecting a magnetic bead and a gold (Au) electrode.

FIGS. 13A-B provide a symbolic representation of logic gates forming a latch.

FIG. 14 schematically depicts electron transport through an antibody.

FIGS. 15A-15D provide experimental results for electron transport through antibodies.

FIG. 16 provides exemplary secondary structures of peptides.

### DETAILED DESCRIPTION

A polypeptide can conduct electricity in a closed circuit. Conformational changes in the polypeptide due to posttranslational modifications or ligand binding can effect the conductive properties of the polypeptide which can be measured. In such a closed circuit, a polypeptide having at least one residue capable of reversible modification can be used as a molecular switch. Circuits comprising such molecular switches can be used, for example, in methods for assessing the modification state of a polypeptide, determining the activity of an enzyme of interest, identifying compounds that affect the activity of an enzyme of interest, storing data, detecting the presence of a compound and identifying inhibitors of protein-protein interactions.

Unless indicated otherwise, all technical and scientific terms are used herein in a manner that conforms to common technical usage. Generally, the nomenclature of this description and the described laboratory procedures, including cell culture, molecular genetics, and nucleic acid chemistry and hybridization, respectively, are well known and commonly employed in the art. Standard techniques are used for recombinant nucleic acid methods, oligonucleotide synthesis, cell culture, tissue culture, transformation, transfection, transduction, analytical chemistry, organic synthetic chemistry, chemical syntheses, chemical analysis, and pharmaceutical formulation and delivery. Generally, enzymatic reactions and purification and/or isolation steps are performed according to the manufacturers' specifications. Absent an indication to the contrary, the techniques and procedures in question are performed according to conventional methodology disclosed. Specific scientific methods relevant to the present invention are discussed in more detail below. However, this discussion is provided as an example only, and does not limit the manner in which the methods of the invention can be carried out.

### DEFINITIONS

The term “molecular switch” is defined as a device for changing the flow of electric current through molecules configured to form an electric circuit.

As used herein, the term “polypeptide” refers to a polymer in which the monomers are amino acids and are joined together through peptide or disulphide bonds. More specifically, “polypeptide” refers to an amino acid chain or a fragment thereof, such as a selected region of protein that is of interest in a binding interaction, or a synthetic amino acid chain, or a combination thereof. A polypeptide can be between about 2 and about 500 amino acids in length, preferably about 4 to about 300, more preferably about 6 to about 200 amino acids, and even more preferably about 10 to about 50 or 100 amino acids in length, most preferably about 10 to about 30 amino acids in length. Additionally, amino acids other than naturally-occurring amino acids, for example β-alanine, phenyl glycine or homoarginine, may be included. Commonly-encountered amino acids which are not gene-encoded may also be used in the present invention. The amino acids of the inventive polypeptides can be either the D- or L-optical isomer.

As used herein, the term “reversible modification” refers to a change to an amino acid than can be repealed such that the amino acid can be returned to its original state. Examples include, but are not limited to, posttranslational modifications, such as phosphorylation, acetylation, glycosylation, alkylation, methylation, hydroxylation, nucleotidylylation, lipidation, biotinylation, glutamylation, glycylation, isoprenylation, sulfation, deamination, ubiquitination, metal chelation, oxidation and side chain modification.

A “control polypeptide” represents either a modified or an unmodified state of a polypeptide. Often, a control peptide represents the original state of a polypeptide. In such a case, control peptide denotes a polypeptide that has not undergone a reversible modification.

The term “latch” is an asynchronous sequential logic device used to store data. One latch is capable of storing one bit of data.

The term “logic gate” is defined as an arrangement of one or more switches used to perform Boolean logic operations.

### DISCUSSION

The electron transport of polypeptides have been studied in circuits using aqueous solutions. As will be described below, polypeptides are capable of conducting electricity in closed circuits where the polypeptide itself is the only means of transmission. In addition, a conformational change of the peptide can affect the conductance of the peptide.

In one embodiment, a closed circuit comprises a molecular switch comprising a polypeptide having at least one residue capable of reversible modification. The polypeptide can be a naturally occurring or non-naturally occurring amino acid chain or a fragment thereof, such as a selected region of a protein that is of interest in a binding interaction, or a synthetic amino acid chain, or a combination thereof. The polypeptide of the molecular switch can be between about 2 to about 500 amino acids in length, preferably about 4 to about 300, more preferably about 6 to about 200 amino acids, even more preferably about 10 to about 50 or 100 amino acids in length and most preferably about 10 to about 30 amino acids in length.

Modifications to the polypeptide can be made enzymatically or chemically. Chemical and/or physical treatment of a polypeptide such as any perturbation to the polypeptide, including exposure to chemicals, denaturants and agents that modify the polypeptide structure as well as exposure to electrical fields, magnetic fields, electromagnetic fields and other forms of energy are used in the design and characterization of polypeptides for specific applications. These modifications affect the polypeptide in numerous ways including chemical and structural modification.

Conferral of posttranslational modifications onto amino acid residues is a specific mechanism for inducing function-altering conformational shifts in a protein's structure. Shifts in the polypeptide's conformation switch the polypeptide into a new functional state. A fundamental mechanism for altering protein conformation and function is phosphorylation. Conformational shifts in a polypeptide of the molecular switch alters the electron transport of the polypeptide. The ability to perform electron transport modification enables polypeptides to be used in molecular closed circuit applications.

A molecular switch comprising a polypeptide can be prepared, for example, by attaching the polypeptide to either a source or drain electrode and connecting the polypeptide to the other electrode so as to form a closed circuit. A polypeptide can be attached to an electrode or immobilized in a number of ways. For example, a molecular circuit device may consist of a pair of source/drain electrodes coated with a conductive Au surface. These surfaces are conjugated to peptides using the sulfur atom contained with in cysteine residues.

An immobilized polypeptide is bound to a solid phase support. This binding may be covalent or via ionic bonding, hydrogen bonding, van-der-waals forces or any other non-covalent attachment, including antibody-antigen attachment, Ni-NTA attachment, avidin-biotin pairing and the use of GST tags. The solid phase may be a membrane, for example supported nitrocellulose, a bead, for example an agarose, glass or sepharose bead, a plastic substrate such as an ELISA dish or other plate, or may be a BIAcore chip or other silicon based chip. In one aspect, the polypeptide can be bound to the support in such a way that it is at least partly free in solution. In another aspect, the polypeptide can be bound to the support via an N- or C-terminal linkage, for example via a C-terminal cysteine residue.

In another aspect, secondary structural motifs, such as the following alpha helical motifs: two, three, and four-stranded coiled-coil motifs; four helix bundles, secondary structural elements that associate with DNA (bZIP, HTH, bHLH, bHLH-ZIP), zinc finger motifs, and helix-loop-helix calcium binding motifs, can be used as the linking moiety between the peptide semiconductor and the electronics interface. Beta pleated sheets may be utilized as well.

In one aspect, the electrode can be gold, in which case the linker motifs can be capped at the amino or carboxyl termini with cysteines. This enables the polypeptide to be directly linked to the gold through covalent linkage. Alternatively, for silicon or other surfaces the material surface may be derivatized with APTES, which provides conjugation sites for amino acid residues.

The polypeptide can be connected to the second electrode of the source/drain circuit described above by operably connecting the source and drain electrode via a molecular junction. Techniques for forming and implementing molecular junctions are described in Long, D. P. et al. Magnetic directed assembly of molecular junctions. Applied Physics Letters 86, 153105 (2005), which is herein incorporated by reference.

Magnetically driven self-assembly is an attractive solution because it provides high-yield, accurate placement and predictable orientation for deposited species. Magnetic entrapment also offers a controlled “bottom-up” self-assembly process that does not require electric fields, e-beam lithography, high temperatures, or individually addressing devices, such as affinity tags, to initiate deposition. Magnetic entrapment is attractive because it can address organic monolayers with a top metal contact without harsh chemicals or processes that may erode or alter the self-assembling monolayer (SAM). Finally, since magnetic entrapment performs well as a parallel technique, this method has the potential to generate a large number of devices simultaneously in a wafer-level assembly process.

For example microspheres of silica (1.5 um diameter), painted with hemispherical coatings of Ni and Au by sequential evaporation, may be magnetically assembled at the source/drain gap, bridging the conductive gap and allowing for a polypeptide to be electrically connected to a source and drain in a molecular circuit. See FIG. 5. Magnetic-directed assembly provides a wafer-level route for the fabrication of molecular junctions and opens up the potential for hybrid complementary metal-oxide semiconductor/molecular electronic applications.

Such metallized silica can be used in various semiconductor/molecular electronic applications. The colloid is dispersed in anhydrous ethanol. At the point of use, the stock solution is diluted into 100 ml of anhydrous ethanol (200 proof) and sonicated for 30 minutes. For each deposition, approximately 10 ml of this dispersed solution is used. Magnetic assembly is performed in a test-tube by immersing the 1×1 cm² peptide-functionalized magnetic array in the solution for 30 minutes while placed in an external 225 Gauss magnetic field oriented parallel with the long axis of the features in the array. The devices are then dried under nitrogen and transferred to a cryogenic vacuum probe station that had been fitted with a parametric analyzer (Agilent 4155B) operated under computer control for electrical (I/V and IETS) analysis.

The source and drain electrodes, now having a molecular junction, are operably connected to a means for producing electrical current or an electrical signal. For example, the source and drain may be operably connected to a voltage source, such as a battery or a signal generator. The molecular circuit may also include various circuit elements including, but not limited to inductors, capacitors, resistors, diodes, etc.

In addition, a means for detecting changes in the electrical signal across the molecular switch can be operably connected to the circuit. The detector can detect voltage (V) and/or current (I) and can have any configuration that enables detection of current/voltage through the polypeptide to be made. For example, changes in the electrical signal across the molecular switch can detected by using an electrical detection method selected from the group consisting of impedance spectroscopy, cyclic voltammetry, AC voltammetry, pulse voltammetry, square wave voltammetry, AC voltammetry, hydrodynamic modulation voltammetry, conductance, potential step method, potentiometric measurements, amperometric measurements. current step method, other steady-state or transient measurement methods, and combinations thereof.

In another embodiment, a sensor comprising a closed circuit includes a polypeptide that undergoes a conformational change upon binding to a ligand, a means for producing an electrical signal and a means for measuring the conductive properties of the polypeptide. In one aspect, the sensors can comprise a closed circuit consisting of a metal-polypeptide-metal junction or an array of probe molecules attached to sensing electrodes. Detecting the presence of a compound, such as an enzyme, or the activity of that compound, is accomplished by measuring the change in the conductive properties of the polypeptide or probe molecule. Ligand binding produces a conformational change resulting in the change of electrical properties.

In one aspect, such a sensor can be deployed in situ to monitor continuously fluctuations in analyte, e.g., in the blood stream of a patient to monitor blood glucose, etc., in water samples to monitor for toxins, pollutants, or in a bioreactor or chemical reactor to monitor reaction progress. In other embodiments, analytes detectable using the sensors include organic and inorganic molecules, including biomolecules. The analyte can be an environmental pollutant (e.g., a pesticide, insecticide, toxin, etc.); a therapeutic molecule (e.g., a low molecular weight drug); a biomolecule (e.g., a protein or peptide, nucleic acid, lipid or carbohydrate, for example, a hormone, cytokine, membrane antigen, receptor (e.g., neuronal, hormonal, nutrient or cell surface receptor) or ligand, or nutrient and/or metabolite such as glucose); a whole cell (including a procaryotic (such as pathogenic bacterium) and eucaryotic cell, including a mammalian tumor cell); a virus (including a retrovirus, herpesvirus, adenovirus, lentivirus, etc.); or a spore.

In another embodiment, a device for storing data comprising at least one latch comprises a first logic gate and a second logic gate wherein each first and second logic gate comprises a molecular switch comprising a polypeptide having at least one residue capable of reversible modification.

As described above, a molecular switch comprises a polypeptide. Modification of the peptide affects the electron transport properties of the peptide. For example, a non-modified peptide may conduct significantly more current (I) than a modified peptide. Thus, by using the polypeptide capable of reverse modification, the molecular circuit becomes a two-state system. Accordingly, a molecular switch having a non-modified polypeptide can be characterized as being in an “ON” state. Conversely, the same molecular switch is characterized as being in an “OFF” state upon modification of the polypeptide. It should be understood that the ON and OFF state discussed above depends on the electron transport characteristics of a polypeptide and can be reversed.

Given a molecular circuit capable of having two states, it follows that the molecular circuit may be used in conjunction with other multi-state molecular circuits to construct logical gates. Logical gates are the basic components of computers and in this specific application may be used to perform biological computations. Biological computation utilizes biological molecules to carry-out general purpose digital computation. Two advantages of biological computation over conventional transistor-based computers is size and energy consumption. Further, biological computation is more suited for carrying out computations in a biological environment.

One or more molecular switches can be oriented within a closed circuit to form a Boolean logic gate such as an AND, OR, NOR or NAND gate. For example, two molecular switches connected in series may act as an AND or NOR logic gate. In the AND gate, for example, current will only flow in the circuit if both molecular switches are set to “ON”. Two molecular switches connected in parallel may act as an OR or NAND gate. For, example, in a closed circuit having an OR gate, current will flow in the circuit if either of the two molecular switches are set to “ON”.

In computing systems NAND and NOR gates are most frequently used. The NAND and NOR gates are known in Boolean logic as universal gates. Universal logic gates can be used to form any other logic function. It follows then that the NAND and NOR logical gates are preferably components for forming a latch. A latch is the simplest form of asynchronous memory device capable of storing one bit of information. Consequently, larger amounts of data may be stored using a plurality of latches. As shown in FIG. 13, two NOR gates or two NAND gates may be cross-coupled to form a latch.

FIG. 13A illustrates a SR latch, where S and R stand for ‘set’ and ‘reset’. The latch is constructed from a pair of cross-coupled NOR (negative OR) logic gates. The stored bit is present on the output marked Q. Normally, in storage mode, the S and R inputs are both low, and feedback maintains the Q and Q outputs in a constant state, with Q the complement of Q. If S (set) is pulsed high while R is held low, then the Q output is forced high, and stays high when S returns low. On the other hand, if R is pulsed high while S is held low, then the Q output is forced low, and stays low when R returns low. If S and R are brought high at the same time, both NOR gates will output zeros, leading to the inconsistent output Q=!Q, so this condition must be avoided.

FIG. 13B illustrates a SR latch where S and R stand for ‘not set’ and ‘not reset’. This is constructed from a pair of cross-coupled NAND (negative AND) logic gates. Operation is similar to that of the SR latch in FIG. 13A, except that the S and R inputs are now active-low instead of active-high.

Multiple latches used in conduction are able to store several bits of information. Storage is a fundamental concept in basic computer design and architecture. Thus, given that the molecular circuit described above can be configured to provide storage, a biological computer can be created using the molecular switch described above.

In one embodiment, there is provided a method for assessing the modification state of a polypeptide comprising attaching the polypeptide to either a source or drain electrode and connecting the polypeptide to the other electrode so as to form a closed circuit; measuring the conductive properties of the polypeptide; and comparing the measured conductive properties to that of one or more control polypeptides. For example, the method can be used to determine the methylation state of polypeptide.

In general, when utilizing the inventive methods described herein, the conductance of the control polypeptide can be measured at the same general time as the polypeptide. Alternatively, it can be measured at a time much earlier, and stored for later use.

In another embodiment, there is provided a method of determining the activity of an enzyme of interest in a sample comprising adding to the sample a polypeptide having at least one residue susceptible to modification by the enzyme; recovering the polypeptide from the sample, attaching it to a source or drain electrode and connecting the polypeptide to the other electrode so as to form a closed circuit; measuring the conductive properties of the polypeptide; and comparing the measured conductive properties to that of one or more control polypeptides. In one aspect, the method can be used to monitor the activity in a sample. In this regard, polypeptide from the sample would be recovered and measured repeatedly over time. In one embodiment, the enzyme is involved in posttranslational modification. For example, the enzyme could be involved in phosphorylation, acetylation, glycosylation, alkylation, methylation, hydroxylation, nucleotidylylation, lipidation, biotinylation, glutamylation, glycylation, isoprenylation, sulfation, deamination, ubiquitination, metal chelation, oxidation or side chain modification.

In another embodiment, there is provided a method of determining the activity of an enzyme of interest in a sample comprising exposing the sample to a closed circuit comprising a molecular switch comprising a polypeptide having at least one residue susceptible to modification by the enzyme; measuring the conductive properties of the polypeptide; and comparing the measured conductive properties to that of one or more control polypeptides. In one aspect, the at least one residue is susceptible to posttranslational modification.

In another embodiment, there is provided a method of identifying compounds that affect the activity of an enzyme of interest, comprising (A) exposing the enzyme to a closed circuit comprising a molecular switch comprising a polypeptide having at least one residue susceptible to modification by the enzyme and measuring the conductive properties of the polypeptide; (B) exposing the enzyme to a test compound; (C) repeating step (A); and (D) comparing the measured conductive properties from steps (A) and (C). In one embodiment, the enzyme is involved in posttranslational modification. For example, the enzyme could be involved in phosphorylation, acetylation, glycosylation, alkylation, methylation, hydroxylation, nucleotidylylation, lipidation, biotinylation, glutamylation, glycylation, isoprenylation, sulfation, deamination, ubiquitination, metal chelation, oxidation or side chain modification. In another embodiment, the enzyme is a kinase.

A method also is provided for storing data comprising providing at least one latch comprising a first logic gate and a second logic gate wherein each first and second logic gate comprises a molecular switch comprising a polypeptide having at least one residue capable of a reversible modification. Methods for creating logic gates comprising a molecular switch comprising a polypeptide are described above.

In another embodiment a method of detecting the presence of a substance in a sample comprising exposing the sample to a sensor comprising a closed circuit comprising (i) a polypeptide that undergoes a conformational change upon binding to a ligand wherein the polypeptide is attached to either a source or drain electrode and connected to the other electrode so as to form a closed circuit, (ii) means for producing an electrical signal; and (iii) means for measuring the conductive properties of the polypeptide; measuring the conductive properties of the polypeptide; and comparing the measured conductive properties to that of one or more control polypeptides.

In another embodiment, there is provided a method for identifying a compound that inhibits binding between a pair of polypeptides, the method comprising attaching a first polypeptide of the pair to a source or drain electrode and connecting the first polypeptide to the other electrode so as to form a closed circuit; exposing a second polypeptide of the pair to a test compound; exposing the second polypeptide to the attached first polypeptide; measuring the conductive properties of the first polypeptide; and comparing the measured conductive properties to that of one or more control polypeptides.

In another embodiment, there is provided a method for identifying a compound that inhibits binding between a pair of polypeptides, the method comprising (A) attaching a first polypeptide of the pair to a source or drain electrode and connecting the first polypeptide to the other electrode so as to form a closed circuit; (B) exposing the attached first polypeptide to a second polypeptide of the pair; (C) measuring the conductive properties of the first polypeptide; (D) exposing the attached first polypeptide to a test compound; (E) measuring the conductive properties of the first polypeptide; and (F) comparing the measured conductive properties from steps (C) and (E).

In another aspect, the ends of protein semiconductors can be used to drive assembly of protein wires. Examples include, but are not limited to, alpha helical association motifs, such as two, three, and four-stranded coiled-coil motifs, four helix bundles, secondary structural elements that associate with DNA (bZIP, HTH, bHLH, bHLH-ZIP), zinc finger motifs, and helix-loop-helix calcium binding motifs. Beta pleated motifs such as immunoglobulin folds also can be used. These elements can be attached to the ends of polypeptide semiconductors through recombinant DNA techniques, through peptide synthesis, or through the bioconjugation of the wires to the polypeptide connecters. Wires with complimentary ends can then be linked through molecular self assembly.

### EXAMPLES

### 1. Phosphate-Based Electron Transport Switch for Protein Semiconductor

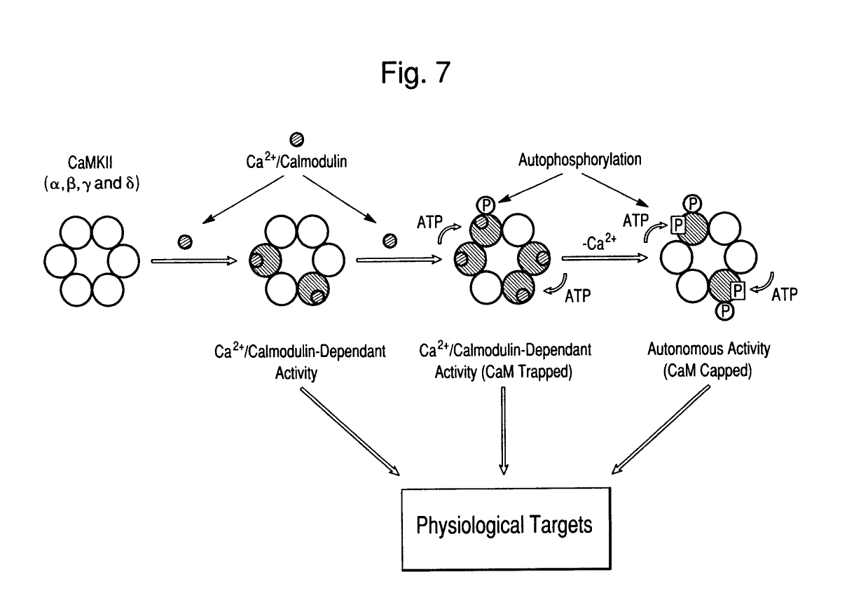

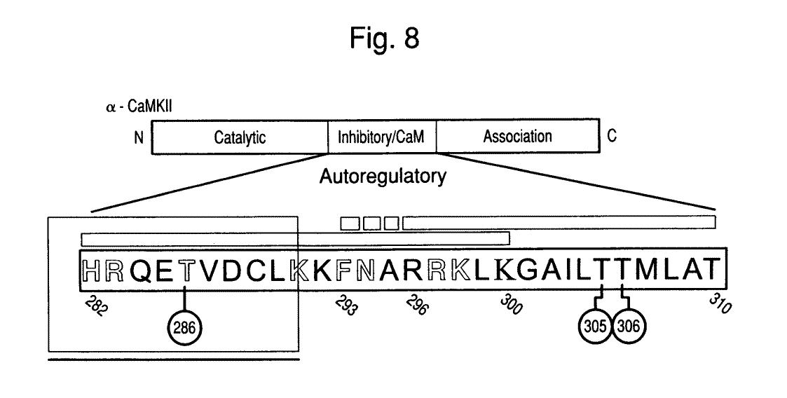

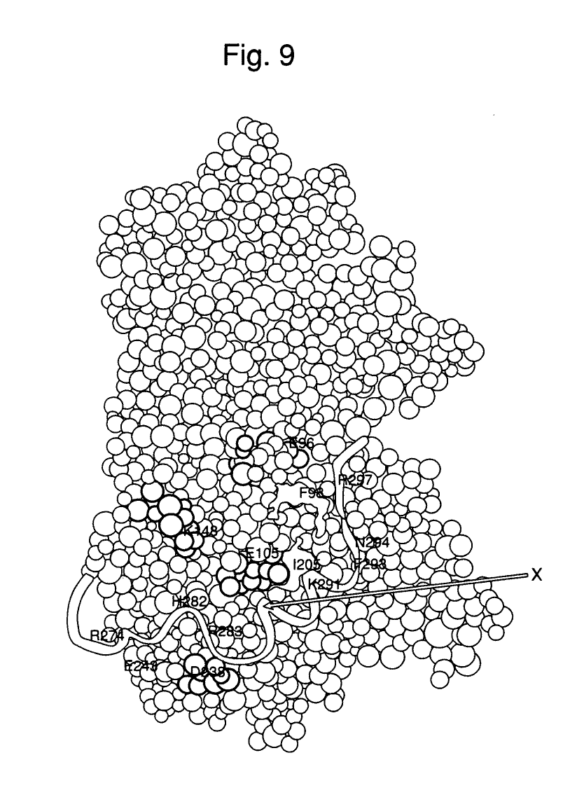

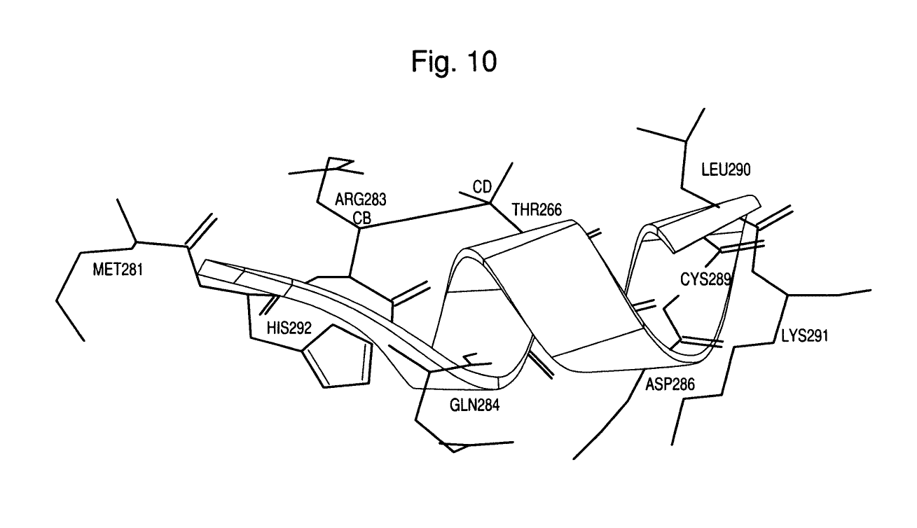

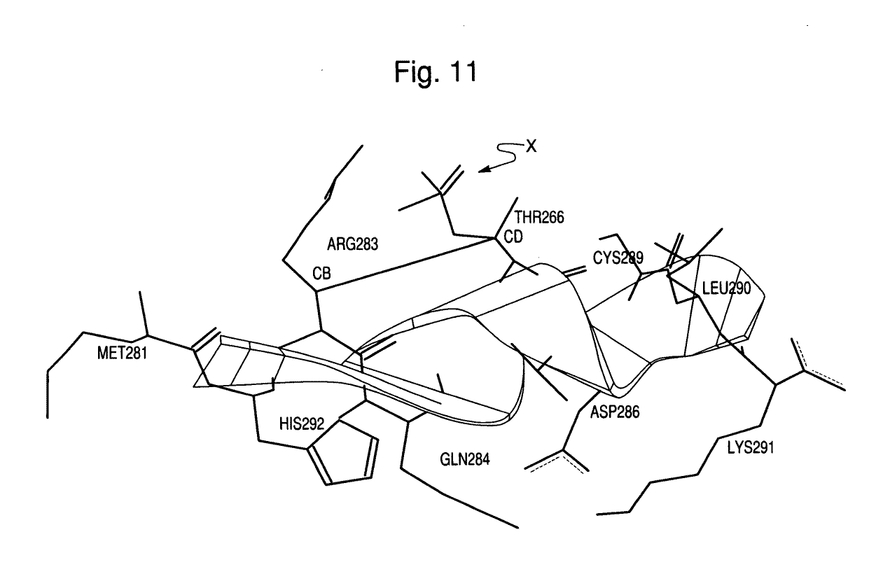

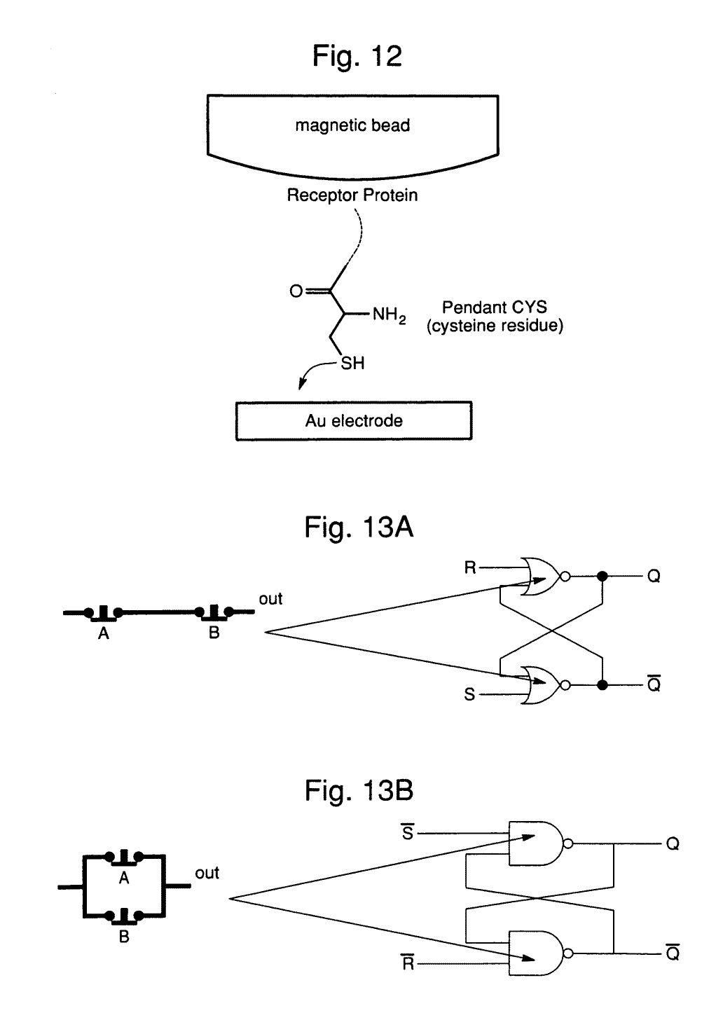

This example provides a structural and electronic evaluation of a two-state alpha-helical polypeptide derived from a well-studied signaling protein, Ca²⁺/Calmodulin (CaM)-dependent protein kinase II (CaM kinase II). CaM Kinase II is an important mediator of Ca²⁺ signaling pathways, which not only phosphorylates itself but also other proteins in order to propagate Ca²⁺ mediated signaling. See FIG. 7. Phosphorylation of Thr 286, within the regulatory segment of the protein, induces a conformational shift that frees this protein from Ca²⁺-mediated activation by preventing the association of the protein's regulatory segment with the kinase domain of the protein, which is intrinsically active.

An eleven amino acid sequence based on a fragment from the regulatory domain of CaM Kinase II (amino acids 281-291) was utilized as the test peptide: Met-His-Arg-Gln-Glu-Thr-Val-Asp-Cys-Leu-Lys. Features of this peptide include: 1) a cysteine residue is present within the sequence, which enables covalent coupling to a gold electrode; 2) a threonine residue is present within the sequence, which is a site for phosphorylation; and 3) in its native state, this region of the protein has an alpha-helical structure and elements of random coil. As a basis for in silico structural studies, the coordinates for amino acids 281-291 were obtained from the crystallographic structure of CaM kinase II. Rosenberg, et al. Cell, 123: 849-860 (2005). After substituting Ile in the position 281 by Met and ASP in position 285 with Glu, energy minimization was performed using conjugate gradient method on the following amino acid sequence: Met-His-Arg-Gln-Glu-Thr-Val-Asp-Cys-Leu-Lys. In the minimized structure the side chain of threonine in position 286 was phosphorylated and the modified peptide was further minimized with the same algorithm. In both cases CHARMm force field was used. MacKerel et al., J. Phys. Chem. B., 102: 3586-3616 (1998). The minimization for both T286 and pT286 peptides was carried out with no solvent and with the implicit solvent represented by distance-dependent dielectric. In the second case the solvent dielectric constant was set to 2.

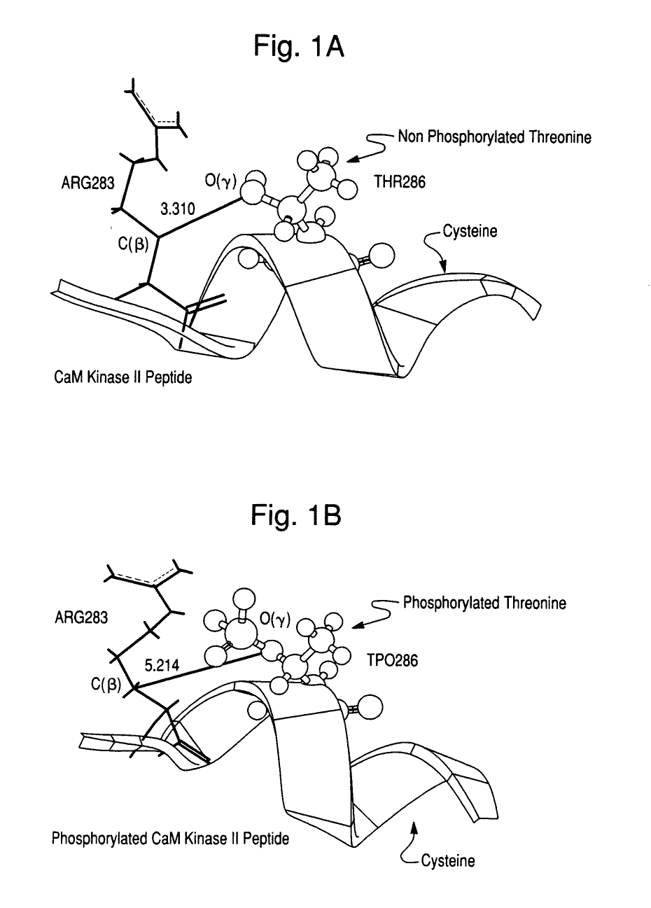

As shown in FIG. 1(a), the modeling studies indicate that the non-phosphorylated peptide is comprised of a combination of random coil and alpha helical secondary structural elements. Recapitulating the x-ray crystallographic finding, Thr 286, the site of phosphorylation, is found within the helical portion of the peptide. The modeled structure for the phosphorylated peptide, see FIG. 1(b), demonstrates a marked shift into a more extended conformation. For example, the inter-atom distance between C(β) of Arg 283 and O(γ) of Thr 286 in the non-phosphorylated peptide is 3.310 Å. In contrast, the inter-atom distance between C(β) of Arg 283 and O(γ) of Thr 286 in the phosphorylated peptide is 5.214 Å, which is an increased distance of 1.904 Å. The finding of a substantially altered peptide conformation is consistent with previous structural studies of this and other phosphorylated polypeptides and proteins.

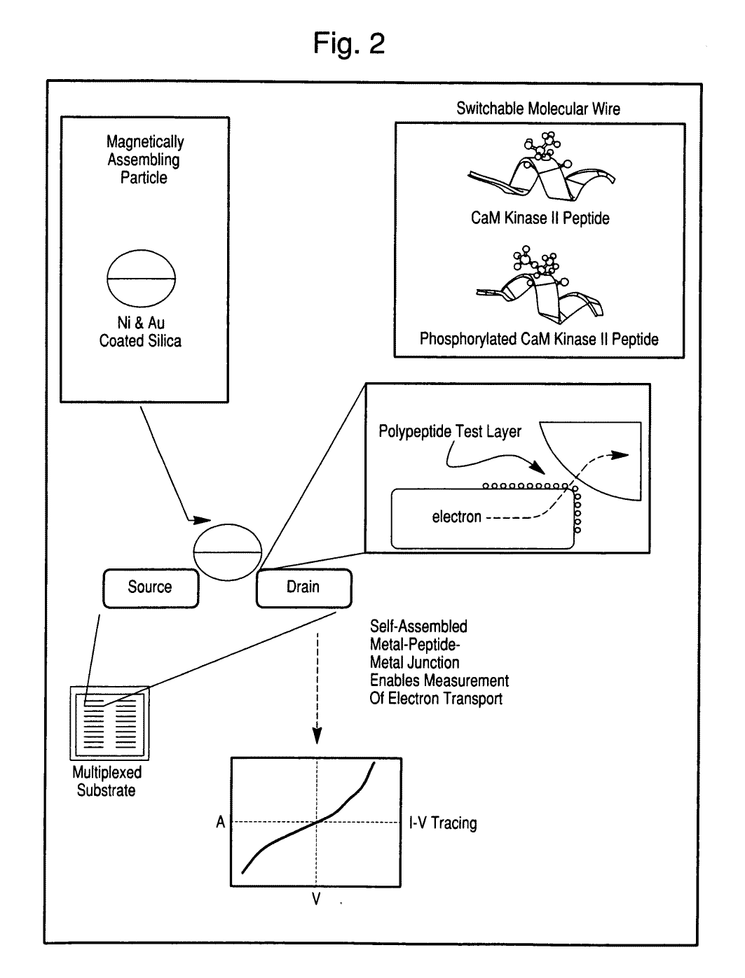

Having identified a molecular switch that alters the conformation of the peptide, electrical studies were performed on the peptides utilizing devices consisting of metal-molecule-metal junctions, as illustrated in FIG. 2. This platform, which is described in detail elsewhere, has been previously utilized to study electron transport through small molecules such as undecanethiol, oligo(phenylene ethynylene), and oligo(phenylene vinylene), but has not been used to study electron transport in peptides. Long, D. P. et al., Applied Physics Letters, 86:153105 (2005).

The electron transport properties of the nonphosphorylated Cam Kinase II derived peptide, shown in FIG. 1a, were tested initially. For immobilization, the cysteine residue within the CaM Kinase II derived peptides used in this study provided an intrinsic linkage mechanism for covalent linkage to the gold-coated electrode. Ulman, A. An Introduction to Ultrathin Organic Films. (Academic Press, San Diego; 1991). Using magnetic assembly, metallized spheres were deposited at the source/drain gap, completing the electrical circuit.

Formation of Self-assembled Monolayers on Magnetic Array

Magnetic arrays composed of evaporated gold-coated nickel and incorporating 0.5 micron spacing between source/drain electrodes were cleaned by sonication in anhydrous tetrahydrofuran (THF) for 10 minutes, followed by UV exposure for 15 minutes, sonication in THF for 10 minutes, in 1,2 dichloroethane (DCE) for 10 minutes, in anhydrous ethanol for 10 minutes, followed by 30% hydrogen peroxide solution exposure for an hour, sonication in anhydrous ethanol for 15 minutes, and final argon plasma cleaning for 10 minutes (Plasma Prep II SPI Supplies, West Chester, Pa.). Gold surface was modified with CaM Kinase (MHRQETVDCLK, Anaspec) and Phosphorylated CaM Kinase (MHRQEpTVDCLK, Anaspec) by incubating the arrays in 2 ml of 10 μM solution in MilliQ water at 4° C. for a duration not less than 24 hours. The substrates were then rigorously rinsed in MilliQ water followed by drying.

Microsphere Junction Fabrication and Measurements

Fabrication of the metallized silica colloid used in the study was previously described. Long, D. P. et al., Applied Physics Letters, 86:153105 (2005). The colloid was dispersed in anhydrous ethanol. At the point of use, the stock solution was diluted into 100 ml of anhydrous ethanol (200 proof) and sonicated for 30 minutes. For each deposition, approximately 10 ml of this dispersed solution was used. Magnetic assembly was performed in a test-tube by immersing the 1×1 cm² peptide-functionalized magnetic array in the solution for 30 minutes while placed in an external 225 Gauss magnetic field oriented parallel with the long axis of the features in the array. The devices were then dried under nitrogen and transferred to a cryogenic vacuum probe station that had been fitted with a parametric analyzer (Agilent 4155B) operated under computer control for electrical (I/V and IETS) analysis. Incorrectly seated assemblies could be detected by metal-metal contacts, which have a characteristic I-V profile, and were easily excluded from the data set (data not shown).

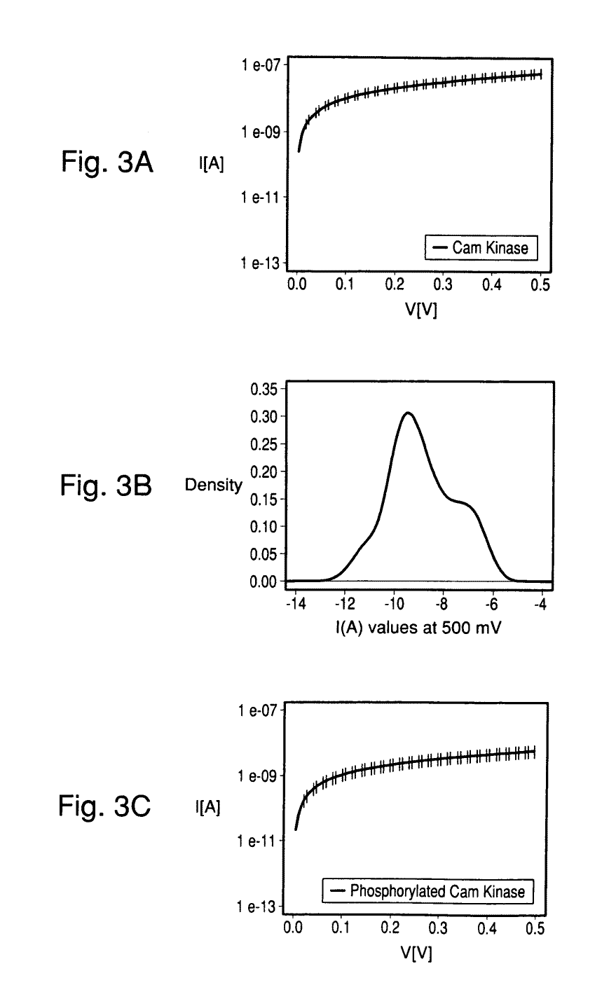

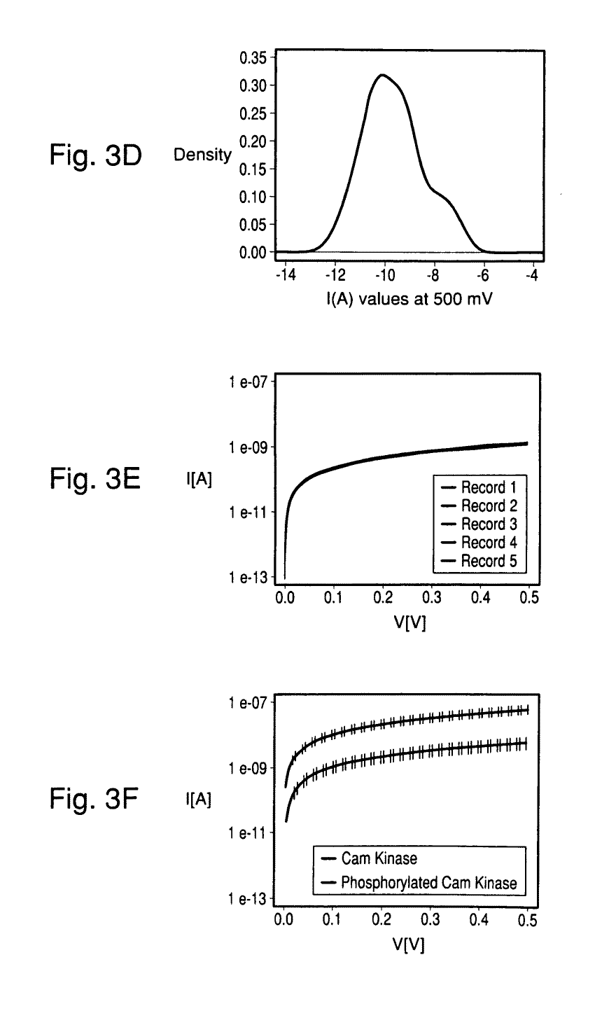

FIG. 3(a) shows the I-V traces (74 total) for the devices coated with non-phosphorylated CaM Kinase II derived peptide, which conducted an average of 62 nA at 0.5 V bias. This average magnitude of electron transport is similar to that previously reported using a similar test configuration for a much smaller organic molecule, oligo(phenylene vinylene). Long, D. P. et al. (2005). Possible mechanisms of transport through alpha helices include: 1) the electrostatic fields created by the dipole moment of peptide helices, 2) π orbitals of the peptide backbone present within helices, and 3) through hydrogen bonds that contribute to the structural stability of helices. Long, D. P. et al. (2005). A relatively uniform arrangement of immobilized peptides is inferred from the unimodal distribution of electron transport behaviors across many devices, as shown in FIG. 3(b).

In order to test the effect of phosphorylation-driven conformational change on electron transport, a phosphorylated form of CaM Kinase II derived peptide was immobilized in order to form a metal-peptide-metal junction. Electron transport through this altered peptide was reproducibly observed in a large number (62 total) of molecular electronics devices as shown in FIG. 3(c), with an average I-V value of 6 nA at 0.5V. As observed with the non-phosphorylated test peptide, a unimodal distribution of electron transport behaviors was observed with the phosphopeptide, shown in FIG. 3(d). Within the same device, the electron transport through the immobilized phosphorylated peptide was highly reproducible as well, see FIG. 3(e). Work by others has demonstrated that helix unfolding impacts the rate of electron transport in a dichromomorphic peptide model, resulting in an order of magnitude difference in electron transport. Fox et al., J. Am. Chem. Soc., 119:5277-5285 (1997). A 10-fold difference in electron transport (see FIG. 3(f)) resulting from the addition of the phosphate group onto the surface of the peptide is likely attributable to a conformational shift within the secondary structure of the peptide. In our molecular model the addition of the phosphate group increased the space between atoms within the peptide. For example, with phosphorylation the distances between the C(β) atoms in Arg 283 and Thr 286 as well as Thr 286 and Cys 289 increased. Further, the overall length of the peptide was increased by 0.323 Å. Shifts in no covalent interactions, such as hydrogen bonding, within the peptide could disrupt intermolecular electron transport mechanisms within the phosphopeptide.

Inelastic Electron Tunneling Spectroscopy

In order to further characterize the molecular devices used in this study, inelastic tunneling spectroscopy was performed, which enables measurement of the vibration spectra of molecules contained in similar test structures. Kushmerick et al., Nano Lett., 4:639-642 (2004); Cai et al. Nano Lett., 5:2365-2372 (2005); Wang et al., Nano Lett., 4:643-646 (2004). IETS studies provide insight into the chemical nature of molecules immobilized at the device site. Performed at 4K, this technique enables the measurement of I/V characteristics as well as first and second harmonics (proportional to dI/dV and d²I/dV²).

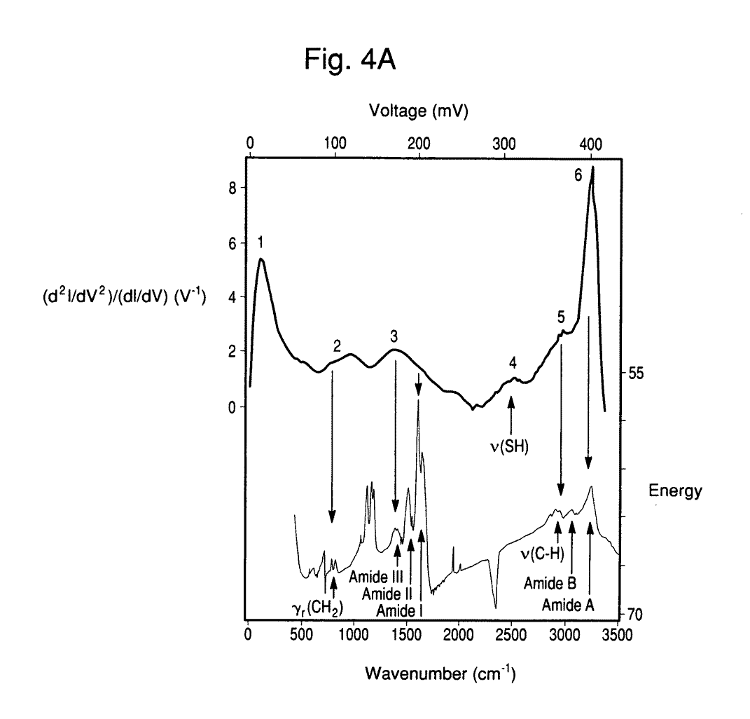

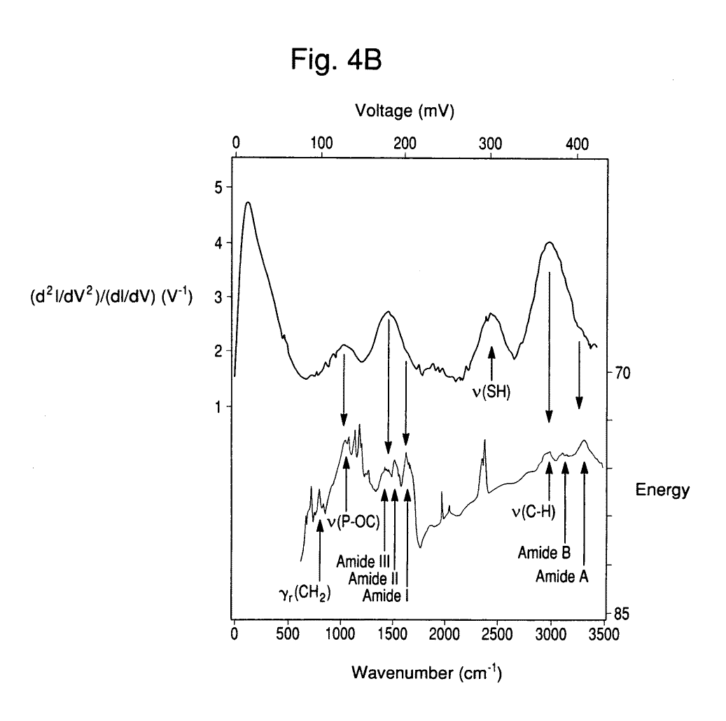

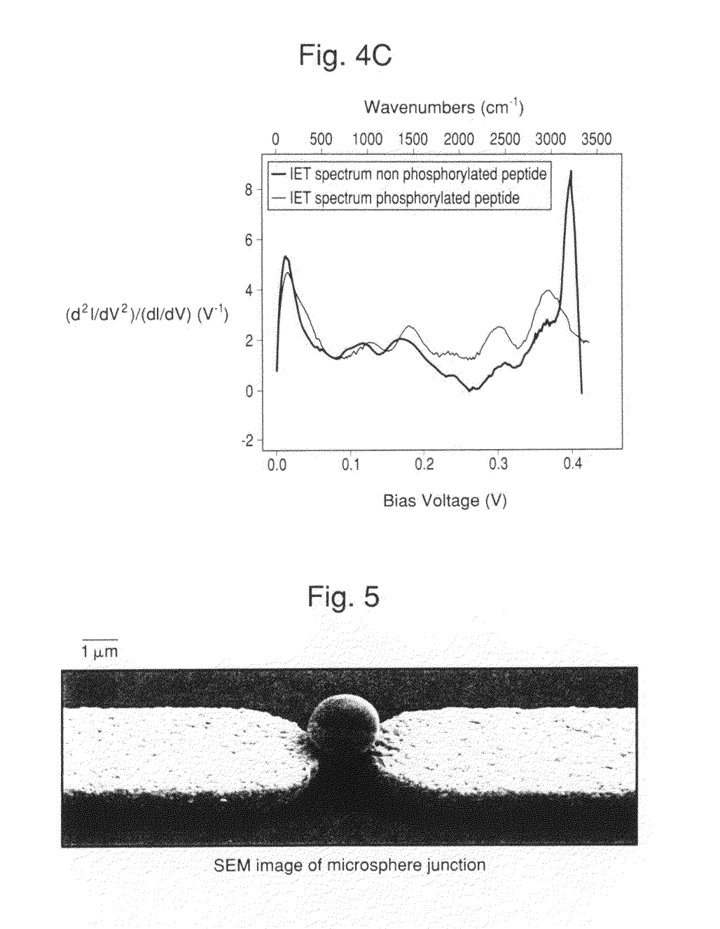

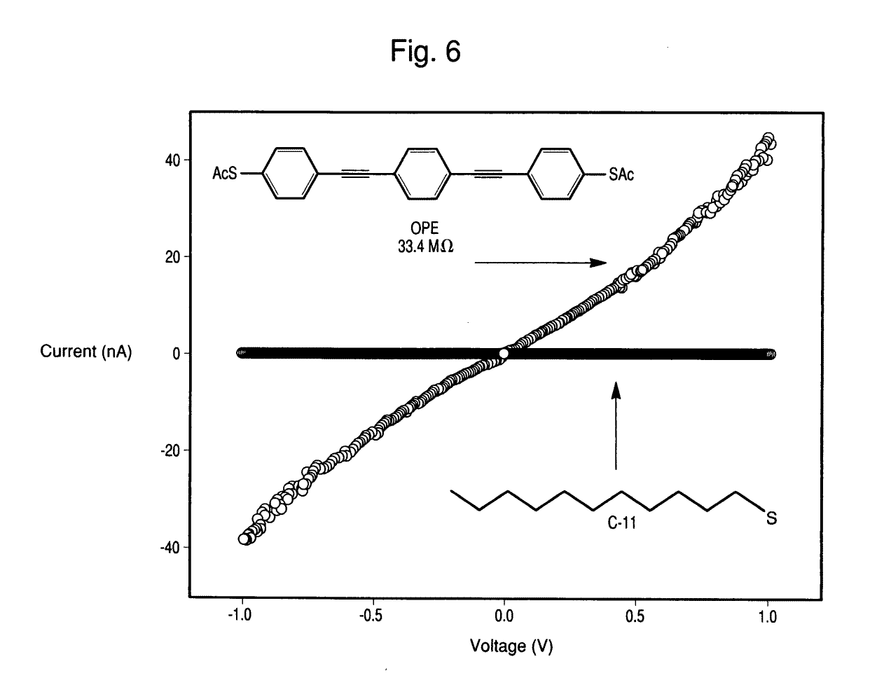

Using devices consisting of microsphere molecular junctions, IETS was acquired as previously described. Kushmerick et al., (2004). In order to accommodate changing current levels and device quality under varying environmental conditions, IETS parameters were optimized for individual. A typical instrument settings is as follows: temperature (4.2 K), lock-in time constant (1 s), ac modulation amplitude (4-10 mV), step size (1-2 mV). In order to enable the lock-in amplifier to stabilize, a delay of 2-4 seconds is applied before each data point is acquired. Each data point is the average of 1000-2000 samples with a delay of ˜1 ms between each. These characteristics are shown, for example, in FIGS. 4(a)-4(c).

The amide I, II, and III bands, which are components of a peptide's backbone structure, are present within both the IETS and FT-IR spectra, as shown in IETS peak #3 Barth, A.; Zscherp, C. Q Rev Biophys 2002, 35, 369-430. IETS peak #6 encompasses a number of peaks detected via FT-IR, including the amide A and amide B bands, also expected constituents of a peptide. Barth et al., (2002). Amino acid side chains appear to contribute to the IET spectra as well. Within IETS peak #3 are expected modes for a number of amino acid side chains, including Asp, Glu, Gln, Arg, Lys, and H is, the peaks of which overlap with each other as well as elements of the peptide backbone. IETS peak #4 accounts for the S—H stretching mode of cysteine, which absorbs in a spectral region free from overlapping by other groups. Barth et al., (2002). The marked vibrational intensity of the IETS S—H vibrational mode, compared to the lack of a prominent FT-IR peak, is attributable to the fact that this functional group is the point of linkage for the peptide to the metal (Au) electrode. All electrons injected into the peptide must exit via the gold-sulfur linkage on the electrode surface making this single bond prominent in IETS.
Phosphate, within the present system, alters electron transport through the test peptide, likely through a conformational shift mechanism. For the phosphopeptide, a peak at 1039 cm⁻¹ consistent with the P—OC stretch within a phosphate group is detected by FT-IR. Silverstein, R. M.; Webster, F. X.; Kiemle, D. J. Spectrometric identification of organic compounds, Seventh ed.; Wiley: Hoboken, N.J., 2005. In the IET spectra, there is a slight red shift in frequency in peak #2 in the non-phosphorylated peptide when compared to the phosphorylated peptide. The shift in the frequency observed in the IETS of the phosphorylated peptide matches the location of the P—OC stretch detected by FT-IR. Taken together, the IETS results for the phosphorylated and non-phosphorylated peptides indicate that the peptide backbone and side chains contribute to electron tunneling in the peptide.

The results demonstrate that a posttranslational modification, i.e., phosphorylation, can be used as a switch to alter electron transport in a polypeptide. Thus, structural modification of polypeptides by the addition of phosphate groups, sugars, nucleic acids, or lipids via enzymatic linkage provides a mechanism for reversibly tailoring the electron transport activity of a nanoelectronics device. For example, a polypeptide containing Ser, Thr, or Tyr residues could be repeatedly phosphorylated/dephosphorylated by cycles of treatments with kinase and phosphatase enzymes, creating a tunable switch for a molecular circuit.

Infrared Spectroscopy

Fourier Transform infrared spectra (FT-IR) were obtained on a Spectrum RX I FT-IR spectrometer (Perkin Elmer, Wellesley, Mass.) at a resolution of 4 cm⁻¹ and at room temperature. Reference spectra of air were recorded and subtracted from the sample spectra. Cam Kinase II derived peptides were dissolved in anhydrous isopropanol at a concentration of 100 μg/ml. The solution was dried on the surface of KBr plates under N₂ and the plates then evaluated.

### 2. Use of an Antibody as a Molecular Switch

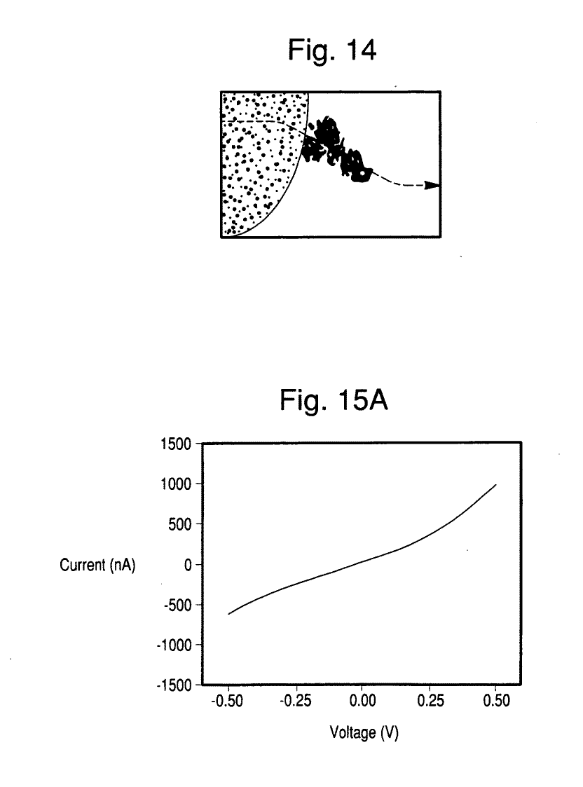

In order to show the applicability of this system to higher order molecular structures, a mature folded protein was immobilized onto the molecular circuit (FIG. 14). A pure mouse monoclonal antibody was utilized as a test protein for the following reasons: a) antibody function is based on the 3-D structure of the variable region b) antibody molecules are a multimer of both heavy and light chains, c) antibodies can recognize peptide ligands with high and low affinities, and d) antibodies are hardy and can be fully functional after being hydrated from a dried state. Thus, the semiconductor properties of an antibody bridging the electrode junction would seem to be one extreme case for our junction array platform. The antibody dimensions are roughly 15-20 nanometers along a side. Although charge transfer through such a long distance in a polypeptide has not been demonstrated previously, our data revealed a current/voltage behavior of the antibody molecule distinct from that found for linker only (see below) as the control, with no evidence of short-circuit artifact.

Several immobilization strategies were available. For example, in one strategy the amino terminal peptide residue is a cysteine. The sulfhydryl group on the cysteine can directly associate with the surface of the gold. This strategy removes the linker moieties required using other linking strategies.

Here, the primary amino group at the amino terminus was linked to the gold using a bioconjugate linker strategy. The gold surface was coated with 1,4-benzenedimethanethiol, yielding a monolayer of exposed thiol groups. These were incubated with a heterobiofunctional linker group Sulfo-GMBS (N-[g-maleimidobutyryloxy]sulfosuccinimide ester). The maleimide group linked to the exposed thiol on the surface of the gold, thereby leaving an NHS group exposed at the surface of the circuit. The peptide was then incubated at an acidic pH, leading to amino terminal linkage to the NHS group.

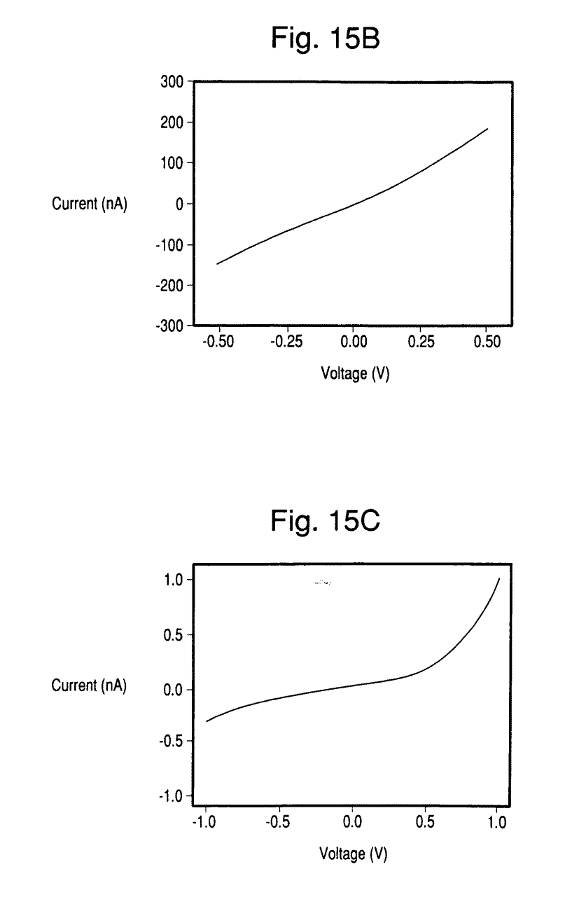

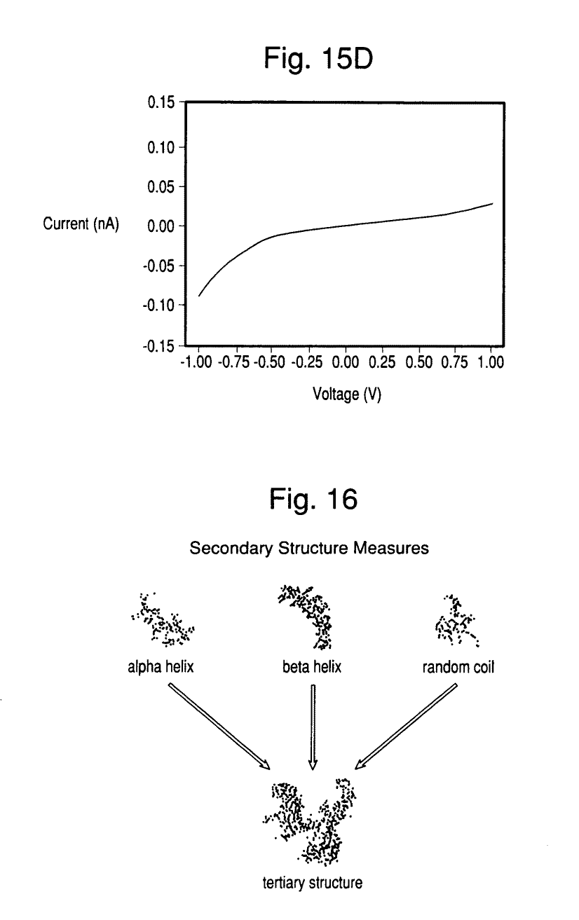

The gold surface of the magnetic array was coated with a heterobifunctional linker with thiol and amino reactive groups. The thiol moiety directed the linker to the gold surface, leaving an exposed amino group that was then treated with glutaraldehyde, a commonly used crosslinking agent. After washing, the antibody was incubated on the substrate. Lysine residues exposed at the surface of the antibody are linkage sites for the glutaraldehyde crosslinker. Because multiple lysine residues are present in a protein of this size, this technique for immobilization potentially leads to a monolayer of antibodies with varying orientations. Once immobilized and thoroughly washed with distilled/deionized water, the surfaces were dried and the circuits completed through the magnetic entrapment of the metallized microspheres. These completed circuits incorporating large, mature proteins, displayed a characteristic electronic current-voltage signature. FIG. 15 shows two examples of these results. Also shown are plots of the current-voltage properties of two individual microsphere bio-junctions incorporating surface-immobilized antibodies (FIGS. 15C and 15D). A large asymmetry is observed in the current-voltage (I-V) traces, suggesting that electron transfer is largely unidirectional through the devices. This is consistent with the fact that the tested microsphere bio-junctions incorporate a covalent gold-thiol (Au—S) bond that immobilizes the antibody to the electrode, while the top contact is a weak non-bonding interaction with the surface of the microsphere. This experiment has demonstrated that current can be made to pass through immobilized antibodies. Varying I-V traces may be due to varying orientation of the antibody. The top two graphs (FIGS. 15A and 15B show control results in which no immobilized antibody is present (linker only). As an extension of these findings, a limited polypeptide sequence was immobilized onto the surface of the molecular circuit (H-Asp-Arg-Val-Tyr-lie-His-Pro-Phe-OH). The primary amino group at the amino terminus was linked to the gold using a bioconjugate linker strategy. The gold surface was coated with a monolayer of thiol groups using a bifunctional thiol linker (1,9-nonanedithiol). These were incubated with a heterobiofunctional linker group Sulfo-GMBS (N-[g-maleimidobutyryloxy]-sulfosuccinimide ester). The maleimide group linked to the exposed thiol on the surface of the gold, thereby leaving an NHS group exposed at the surface of the circuit. The peptide was then incubated at an acidic pH, leading to amino terminal linkage to the NHS group. Current was applied to the completed molecular circuit either with linker alone or linker with peptide. The peptide yielded a current/voltage tracing (FIG. 15) similar to that seen with the antibody (results representative of two peptide tracings and four linker-only tracings). Electric current traveled through peptide/linker combination, even though the linker contained a chain of 9 carbons in sequence.

Current-voltage (I-V) characteristics and conductance measurements were obtained using an Agilent 4155B parametric analyzer (Agilent, Palo Alto, Calif.) under computer control. Magnetic arrays incorporating completed microsphere bio-junctions were placed in the controlled environmental sample chamber of a Desert Cryogenics Semiconductor Vacuum Probe Station. All measurements were performed at room temperature in a nitrogen atmosphere and compared to measurements made in a humidified environment.

The presence of phosphate groups appended to side chains of amino acids are of particular importance in signal transduction. A number of amino acid residues were decorated with phosphate groups, including glycine, arginine, threonine, serine, lysine, tyrosine, and histidine.

The preliminary current profiles obtained show that voltage increments can be made small enough to have very continuous current profiles. Moreover, the current profiles for control and the profiles in the presence of antibody show significant differences of intensity, and differences in the behavior of first derivative and curvature as well (especially in the initial and final sections of the profile).

While the invention is described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention.

1. A closed circuit comprising a molecular switch comprising a polypeptide having at least one residue capable of reversible modification.

2. The closed circuit of claim 1 further comprising
(A) means for producing an electrical signal; and
(B) means for detecting changes in the electrical signal across the molecular switch.

3. The closed circuit of claim 1 further comprising
(A) a signal generator; and
(B) a detector for detecting changes in the electrical signal across the molecular switch.

4. The closed circuit of claim 1 wherein said polypeptide is a CaM kinase II polypeptide.

5. The closed circuit of claim 1 wherein said polypeptide is Met-His-Arg-Gln-Glu-Thr-Val-Asp-Cys-Leu-Lys.

6. A device for storing data comprising at least one latch comprising a first logic gate and a second logic gate wherein each first and second logic gate comprises a molecular switch comprising a polypeptide having at least one residue capable of a reversible modification.

7. The device of claim 6, wherein the reversible modification is a post-translational modification.

8. The device of claim 7, wherein the post-translation modification is selected from the group consisting of phosphorylation, acetylation, glycosylation, alkylation, methylation, hydroxylation, nucleotidylylation, lipidation, biotinylation, glutamylation, glycylation, isoprenylation, sulfation, deamination, ubiquitination, metal chelation, oxidation and side chain modification.

9. (canceled)

10. A sensor comprising a closed circuit comprising (i) a polypeptide attached to either a source or drain electrode and connected to the other electrode so as to form a closed circuit, wherein the polypeptide undergoes a conformational change upon binding to a ligand, (ii) means for producing an electrical signal; and (iii) means for measuring the conductive properties of the polypeptide.

11. The sensor of claim 10, wherein the polypeptide is an antibody.

12.-23. (canceled)

24. A method for storing data comprising providing at least one latch comprising a first logic gate and a second logic gate wherein each first and second logic gate comprises a molecular switch comprising a polypeptide having at least one residue capable of a reversible modification.

25. A method of detecting the presence of a substance in a sample comprising
(A) exposing the sample to a sensor comprising a closed circuit comprising (i) a polypeptide that undergoes a conformational change upon binding to a ligand wherein the polypeptide is attached to either a source or drain electrode and connected to the other electrode so as to form a closed circuit, (ii) means for producing an electrical signal; and (iii) means for measuring the conductive properties of the polypeptide;
(B) measuring the conductive properties of the polypeptide; and
(C) comparing the measured conductive properties to that of one or more control polypeptides.

26.-27. (canceled)


SEQUENCE LISTING (4 sequences)

SEQ ID NO: 1 - protein, 11 aa, Artificial Sequence
  Description of Artificial Sequence Synthetic peptide
MHRQETVDCLK

SEQ ID NO: 2 - protein, 11 aa, Artificial Sequence
  Description of Artificial Sequence Synthetic peptide
MHRQETVDCLK

SEQ ID NO: 3 - protein, 8 aa, Artificial Sequence
  Description of Artificial Sequence Synthetic peptide
DRVYIHPF

SEQ ID NO: 4 - protein, 29 aa, Artificial Sequence
  Description of Artificial Sequence Synthetic peptide
HRQETVDCLKKFNARRKLKGAILTTMLAT